(19) United States
(12) Patent Application Publication
(10) Pub. No.: US 2007/0088403 A1
(43) Pub. Date: Apr. 19, 2007

(54) Methods and systems for establishing parameters for neural stimulation

(76) Inventors: Allen Wyler, Seattle, WA (US); Bradford Evan Gliner, Sammamish, WA (US); Leif R. Sloan, Seattle, WA (US)

Correspondence Address: PERKINS COIE LLP;PATENT-SEA, P.O. BOX 1247, SEATTLE, WA, 98111-1247, US

(21) Appl. No.: 11/254,240

(22) Filed: Oct. 19, 2005

(51) Int. Cl. A61N 1/18

(52) U.S. Cl. 607/45

(57) ABSTRACT

Methods and systems for establishing parameters for neural stimulation are disclosed. A method in accordance with one embodiment includes applying a first stimulus to a first neural population associated with a first neural function, using a first set of stimulation parameters. The method can further include detecting a response to the first stimulus at least proximate to the patient's central nervous system and, based at least in part on the response to the first stimulus and on the first set of stimulation parameters, applying a second stimulus to a second neural population. The second neural population can be associated with a second neural function different than the first neural function and can be stimulated using a second set of stimulation parameters. In a further embodiment, evidence of a neural activity can be detected at the patient's central nervous system, and electromagnetic stimulation of a target neural population at the patient's central nervous system can be automatically triggered, based at least in part on the detected evidence.

### TECHNICAL FIELD

The present invention in directed generally toward methods and systems for establishing parameters for neural stimulation, including techniques for applying neural stimulation parameters from a first neural population having a first neural function to a second neural population having a second neural function different than the first.

### BACKGROUND

A wide variety of mental and physical processes are controlled or influenced by neural activity in particular regions of the brain. In some areas of the brain, such as in the sensory or motor cortices, the organization of the brain resembles a map of the human body; this is referred to as the “somatotopic organization of the brain.” There are several other areas of the brain that appear to have distinct functions that are located in specific regions of the brain in most individuals. For example, areas of the occipital lobes relate to vision, regions of the left inferior frontal lobes relate to language in the majority of people, and regions of the cerebral cortex appear to be consistently involved with conscious awareness, memory, and intellect. This type of location-specific functional organization of the brain, in which discrete locations of the brain are statistically likely to control particular mental or physical functions in normal individuals, is herein referred to as the “functional organization of the brain.”

Many problems or abnormalities with body functions can be caused by damage, disease and/or disorders of the brain. A stroke, for example, is one very common condition that damages the brain. Strokes are generally caused by emboli (e.g., obstruction of a vessel), hemorrhages (e.g., rupture of a vessel), or thrombi (e.g., clotting) in the vascular system of a specific region of the cortex, which in turn generally causes a loss or impairment of a neural function (e.g., neural functions related to face muscles, limbs, speech, etc.). Stroke patients are typically treated using physical therapy to rehabilitate the loss of function of a limb or another affected body part. For most patients, little can be done to improve the function of the affected limb beyond the recovery that occurs naturally without intervention.

One existing physical therapy technique for treating stroke patients constrains or restrains the use of a working body part of the patient to force the patient to use the affected body part. For example, the loss of use of a limb is treated by restraining the other limb. Although this type of physical therapy has shown some experimental efficacy, it is expensive, time-consuming and little-used. Stroke patients can also be treated using physical therapy plus adjunctive therapies. For example, some types of drugs, including amphetamines, increase the activation of neurons in general. These drugs also appear to enhance neural networks. However, these drugs may have limited efficacy because mechanisms by which they operate are very non-selective and they cannot be delivered in high concentrations directly at the site where they are needed. Still another approach is to apply electrical stimulation to the brain to promote the recovery of functionality lost as a result of a stroke. While this approach has been generally effective, it has not adequately addressed all stroke symptoms.

In addition to the motor-related symptoms described above, stroke patients may also suffer from cognitive defects. For example, patients may suffer from neglect, a defect that causes patients to lose cognizance of portions of their surroundings and/or themselves. In other cases, patients may suffer from other cognitive defects, such as memory loss or loss of reasoning ability, in connection with a stroke or other event that causes neural damage. While electromagnetic stimulation has been proposed generally to address cognitive defects, the application of such techniques may in some cases be difficult because, unlike motor neurons which can immediately indicate activation by a corresponding muscle action, cognitive and other non-motor neurons typically do not provide such a readily discernable indication of activation. Accordingly, there is a need to improve the manner in which stimulation is applied to cognitive and other non-motor neurons.

### BRIEF DESCRIPTION OF THE DRAWINGS

FIG. 1A is a schematic view of neurons.

FIG. 1B is a graph illustrating firing an “action potential” associated with normal neural activity.

FIG. 1C is a flow chart illustrating a method for applying stimuli to different neural populations in accordance with an embodiment of the invention.

FIG. 2 is a side elevation view of a human brain illustrating prominent brain structures and representative stimulation sites in accordance with an embodiment of the invention.

FIG. 3 is a partially schematic illustration of a stimulation device configured in accordance with an embodiment of the invention.

FIG. 4 illustrates a stimulation device operatively coupled to an external controller in accordance with another embodiment of the invention.

FIG. 5 is a schematic illustration of a pulse system configured in accordance with an embodiment of the invention.

FIG. 6 is an isometric illustration of a device that carries electrodes in accordance with another embodiment of the invention.

FIG. 7 is a partially schematic, side elevation view of an electrode configured to deliver electromagnetic stimulation to a subcortical region in accordance with an embodiment of the invention.

FIG. 8 is a partially schematic, isometric illustration of a magnet resonance chamber in which the effects of neural stimulation may be evaluated.

FIG. 9 illustrates a patient wearing a network of electrodes positioned to detect brain activity in accordance with further embodiments of the invention.

FIG. 10 is a flow chart illustrating a method for estimating a stimulation parameter for a non-motor neural population based at least in part on information from a motor neural population in accordance with another embodiment of the invention.

FIG. 11 is a flow diagram illustrating a method for providing stimulation to a patient using a set of stimulation parameters selected based at least in part on a response received from the patient's central nervous system.

FIG. 12 is a flow diagram illustrating a method for automatically triggering electromagnetic stimulation based on evidence detected at least proximate to the patient's central nervous system in accordance with yet another embodiment of the invention.

FIG. 13 is a partially schematic illustration of a device that includes both a detector and a stimulator for a patient's central nervous system.

### DETAILED DESCRIPTION

A. Introduction

The present invention is directed generally toward methods and systems for establishing stimulation parameters for neural stimulation processes. In particular embodiments, the methods and systems are directed to establishing stimulation parameters for non-motor and/or non-sensory neurons. In still further embodiments, the stimulation parameters selected for non-motor and/or non-sensory neurons can be based at least in part on stimulation parameters established for motor and/or sensory neurons.

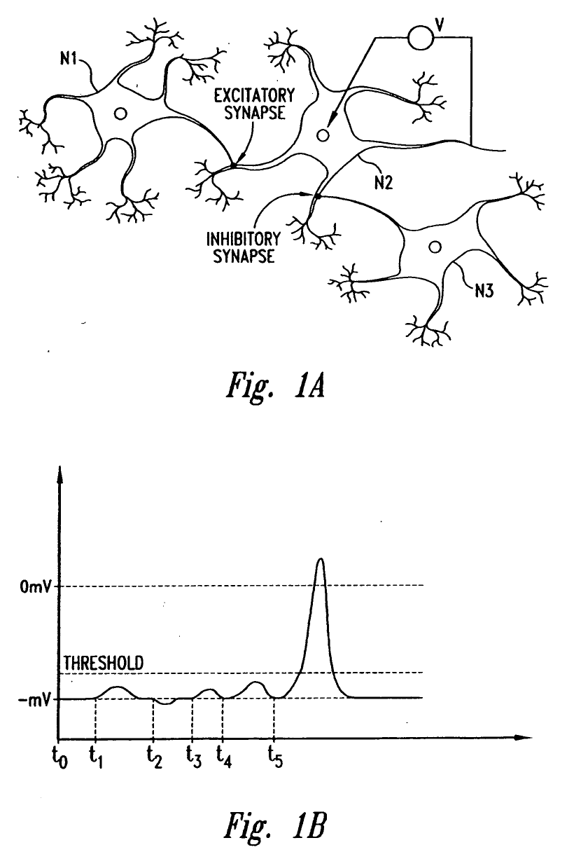

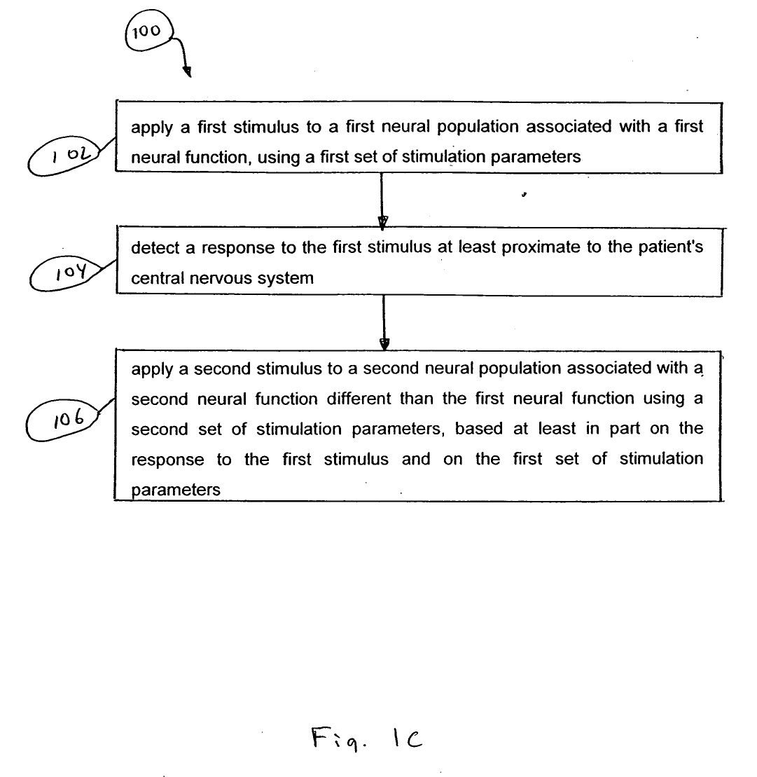

FIG. 1A is a schematic representation of several neurons N1-N3 and FIG. 1B is a graph illustrating an “action potential” related to neural activity in a normal neuron. Neural activity is governed by electrical impulses generated in neurons. For example, neuron N1 can send excitatory inputs to neuron N2 (e.g., at times t₁, t₃ and t₄ in FIG. 1B), and neuron N3 can send inhibitory inputs to neuron N2 (e.g., at time t₂ in FIG. 1B). The neurons receive/send excitatory and inhibitory inputs from/to a population of other neurons. The excitatory and inhibitory inputs can produce “action potentials” in the neurons, which are electrical pulses that travel through neurons by changing the flux of sodium (Na) and potassium (K) ions across the cell membrane. An action potential occurs when the resting membrane potential of the neuron surpasses a threshold level. When this threshold level is reached, an “all-or-nothing” action potential is generated. For example, as shown in FIG. 1B, the excitatory input at time t₅ causes neuron N2 to “fire” an action potential because the input exceeds the threshold level for generating the action potential. The action potentials propagate down the length of the axon (the long portion of the neuron that makes up nerves or neuronal tracts) to cause the release of neurotransmitters from that neuron that will further influence adjacent neurons.

In many instances, it may be desirable to electrically stimulate neurons at subthreshold levels. For example, it may be desirable to provide stimulation to motor neurons at subthreshold levels, and then rely on the (perhaps limited) ability of the neuron to supplement the stimulation signal. The combination of the external electrical stimulation and the neuron's internal or intrinsic ability to generate at least some increase in potential can be enough to exceed the threshold level and generate an action potential. In such instances, it can be important to determine, approximately determine, or estimate what the threshold potential for a given neural population is. Otherwise, the target neurons may be overstimulated, or the neurons may not receive a therapeutically useful dose of stimulation (e.g., if the stimulation is provided outside of a particular stimulation parameter range). In particular instances, however, it may be desirable to briefly stimulate neurons with near threshold, threshold, and/or suprathreshold pulses or bursts, possibly in association with subthreshold stimulation.

In the case of motor neurons, a threshold level can generally be readily determined by varying a stimulation parameter (e.g., increasing a voltage, current, and/or frequency of the stimulation signal) until a motor response is detected. The motor response can often be detected by simply observing or measuring (e.g., using electromyography (EMG)) a muscle action exhibited by the patient. In a generally similar manner, particular sensory neurons can be stimulated and a threshold for such neurons can be detected when the patient receives, reports, or becomes aware of a corresponding sensation. However, for at least some neurons, it may be difficult to detect when the threshold level is exceeded because the patient neither displays an outward action nor reports a sensation. This difficulty can arise, for example, when stimulating neurons associated with cognitive function; or more generally, when stimulating neurons that may be associated with patient functions or responses that are difficult and/or time consuming to readily observe or measure. Such neurons are referred to herein as “silent” neurons.

A method in accordance with one aspect of the invention includes applying a first stimulus to a first neural population associated with a first neural function (e.g., a motor function), using a first set of stimulation parameters. The method can further include detecting a response to the first stimulus at least proximate to the patient's central nervous system. The method can still further include applying a second stimulus to a second neural population associated with a second neural function (e.g., a cognitive function) different than the first neural function using a second set of stimulation parameters, based at least in part on the response to the first stimulus and on the first set of stimulation parameters. In a particular instance, detecting a response to the first stimulus can include detecting a response that is also exhibited by the second neural population. The response can be detected by detecting electrical signals transmitted by the central nervous system, by detecting a change in cerebral blood flow, and/or by detecting a change in a quantity that depends upon cerebral blood flow or upon cerebral blood oxygen levels.

A method for treating a patient in accordance with another aspect of the invention can include directing an electrical signal having a first set of stimulation parameters to a target neural population via an electrode. The method can further include detecting a response to the electrical signal at least proximate to the patient's central nervous system, and changing a value of at least one stimulation parameter of the electrical signal, at least until the response reaches a preselected level. A second set of stimulation parameters can then be selected based at least in part on the value of the stimulation parameter associated with the preselected level. The method can further include directing additional electrical signals to the patient in accordance with the second set of stimulation parameters. Accordingly, the foregoing method need not include stimulation of two different types of neural populations, but can instead rely (at least in part) on responses detected at least proximate to the patient's central nervous system.

A method in accordance with still a further aspect of the invention can include detecting evidence of a neural activity (with the evidence being detected at least proximate to the patient's central nervous system), and then automatically triggering electromagnetic stimulation of a target neural population at the patient's central nervous system, based at least in part on the detected evidence. In particular embodiments, the method can include detecting evidence of a patient's attempt(s) to engage in a neural activity. Accordingly, the foregoing method (and systems that perform the method) can autonomously trigger electromagnetic stimulation at one or more times when the stimulation may be most therapeutic for and/or helpful to the patient carrying out a particular task (e.g., a motor task or cognitive task) that may facilitate the restoration and/or development of a neural function.

B. Methods for Establishing Stimulation Parameters. Including Stimulation Parameters for Diverse Neural Populations

FIG. 1C is a flow diagram illustrating a process 100 for treating a patient in accordance with an embodiment of the invention. The process 100 can include applying a first stimulus to a first neural population associated with a first neural function, using a first set of stimulation parameters (process portion 102). As used in this context, “associated” refers generally to neurons whose activity correlates with a particular neural function. Accordingly, such neurons can be (but need not be) directly or indirectly responsible for executing the function. For example, process portion 102 can include applying an electrical stimulation to a motor neuron using a selected current, voltage, and waveform. In process portion 104, the method can include detecting a response to the first stimulus at least proximate to the patient's central nervous system. For example, process portion 104 can include detecting a change in electrical signals generated by the first neural population, or a change in hemodynamic properties of the blood proximate to the first neural population. Hemodynamic properties can include blood flow levels or blood volume proximate to the first neural population, or a change in a chemical species level (e.g., corresponding to an oxygenation level) of the blood.

Process portion 106 can include applying a second stimulus to a second neural population associated with a second neural function different than the first neural function. For example, process portion 106 can include applying a second stimulus to a cognitive, neuropsychological, neuropsychiatric, or other “silent” neuron. The second stimulus can be applied using a second set of stimulation parameters, the selection of which is based at least in part on the response to the first stimulus and on the first set of stimulation parameters. For example, if the first set of stimulation parameters have a desired relationship relative to the threshold level of the first neural population, then the second set of stimulation parameters can be selected based at least in part on the first stimulation parameters, so as to produce a similar (or calculatedly different) relationship relative to an expected threshold level for the second neural population. In a particular embodiment, a practitioner can determine one or more parameters corresponding to the threshold level of stimulation for a motor neuron, and can interpolate or extrapolate this data to provide a corresponding threshold or non-threshold level of stimulation for a non-motor neuron. In a further particular embodiment, the practitioner can select values for one or more parameters in a manner expected to provide stimulation at between 10% and 90% (e.g., between approximately 25% and 75%, or at approximately 50%) of the threshold value for the non-motor neuron, based on data obtained from stimulation of a motor neuron. If the threshold level is expected to change (e.g., drift) during the course of treatment, the practitioner can update the stimulation parameters accordingly. This function can also be performed automatically in some embodiments.

In another embodiment, if it is determined that stimulating the first neural population with the first set of stimulation parameters produces a desired or beneficial result, some or all aspects of the second set of stimulation parameters (applied to the second neural population) can be selected to be at least approximately identical to the first set of stimulation parameters. A beneficial result in the case of a motor neural population may be the patient's increased ability to perform a motor task. When the same or a similar stimulation parameter is used to stimulate a cognitive neural population, the beneficial result may be the patient's increased ability to perform a cognitive task.

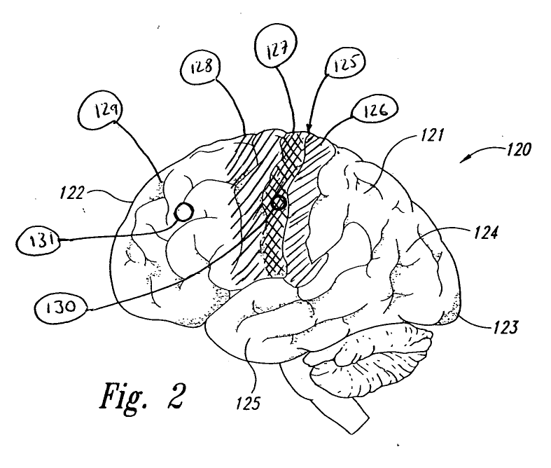

FIG. 2 is a partially schematic illustration of the left side of a human brain 120 illustrating the four major brain lobes, e.g., the parietal lobe 121, the frontal lobe 122, the occipital lobe 124 (which includes the visual cortex 123), and the temporal lobe 125. The parietal lobe 121 and the frontal lobe 122 are separated by the central sulcus 125, with the precentral gyrus (or primary motor cortex) 127 located anterior to the central sulcus, and the postcentral gyrus (or primary somatosensory cortex) 126 located posterior to the central sulcus. Stimulation provided at the primary motor cortex 127 can produce a motor response, and stimulation provided at the primary somatosensory cortex 126 can provide a sensory response in the patient. Also shown are the premotor cortex 128, positioned anterior to the primary motor cortex 127, and the prefrontal cortex 129, positioned anterior to the premotor cortex.

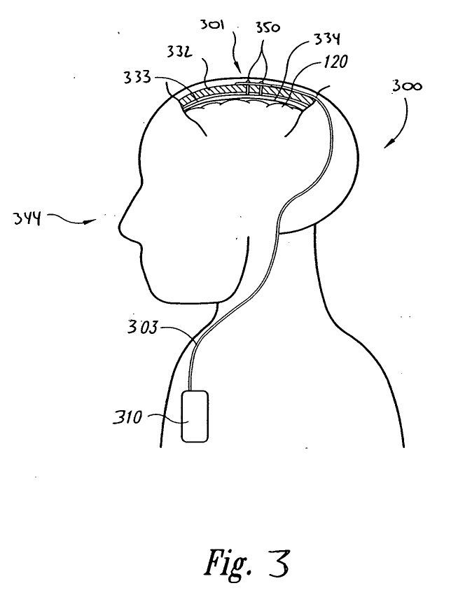

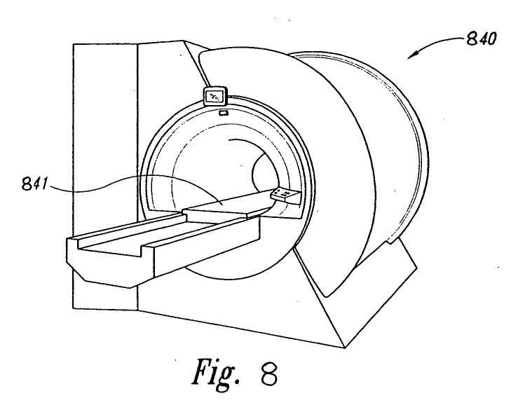

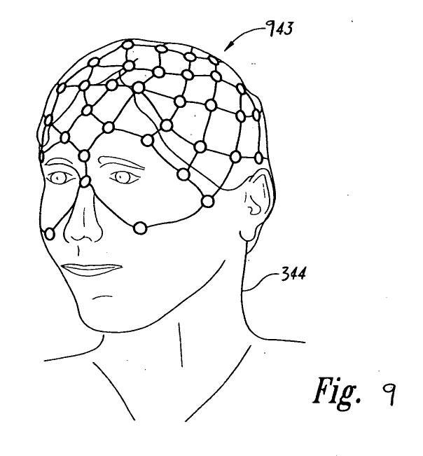

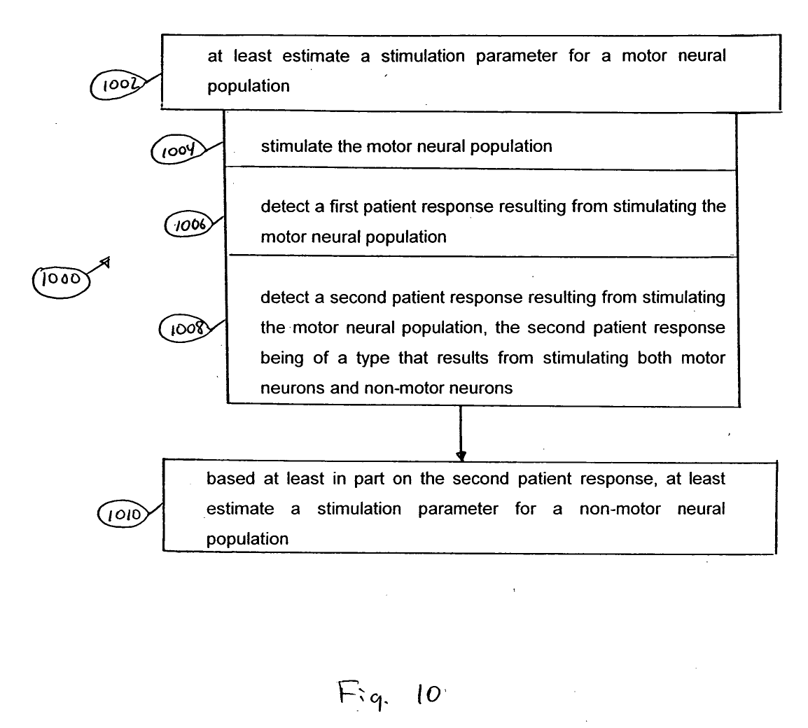

In some instances, it may be desirable to stimulate the prefrontal cortex 129, for example, to provide a cognitive or neuropsychological, neuropsychiatric, and/or other benefit to the patient. However, as described above, it may not be immediately apparent what stimulation parameters should be used to produce the desired beneficial effect because (a) the patient may not exhibit a readily ascertainable external response indicating when the threshold level is closely approached, reached, or exceeded, and/or (b) it may require a significant period of time to determine whether the stimulation produces long-lasting cognitive benefits to the patient. Accordingly, a practitioner can first provide stimulation to a first neural population 130 located at the primary motor cortex 127 to identify stimulation parameters that can then be applied to a second neural population 131 located at the prefrontal cortex 129. FIGS. 3-7 (described below) illustrate devices that can be used to apply the stimulus to the first neural population and/or the second neural population 131. FIGS. 8 and 9 (also described below) illustrate devices that can be used to detect responses to the stimuli provided by these devices.

C. Applying Electrical Stimulation

FIGS. 3-7 illustrate representative devices for applying electrical stimulation. These devices can be located at a first stimulation site to provide stimulation to the first neural population 130 (described above with reference to FIG. 2) using the first set of stimulation parameters. Once the second set of stimulation parameters is determined (based on results from stimulating the first neural population 130), the same or similar devices located at a second stimulation site can provide stimulation to the second neural population 131 (FIG. 2). FIG. 3 is a schematic illustration of a neurostimulation system 300 implanted in the patient 344 to provide stimulation in accordance with several embodiments of the invention. The system 300 can include an electrode device 301 carrying one or more electrodes 350. The electrode device 301 can be positioned in the skull 332 of the patient 344, with the electrodes 350 positioned to stimulate target areas of the brain 120. For example, the electrodes 350 can be positioned just outside the dura mater 333 (which surrounds the brain 120) to stimulate cortical tissue. In another embodiment described later with reference to FIG. 7, an electrode can penetrate the dura mater 333 to stimulate subcortical tissues. In still further embodiments, the electrodes 350 can penetrate the dura mater 333 but not the underlying pia mater 334, and can accordingly provide stimulation signals through the pia mater 334.

The electrode device 301 can be coupled to a pulse system 310 with a communication link 303. The communication link 303 can include one or more leads, depending (for example) upon the number of electrodes 350 carried by the electrode device 301. The pulse system 310 can direct electrical signals to the electrode device 301 to stimulate target neural tissues.

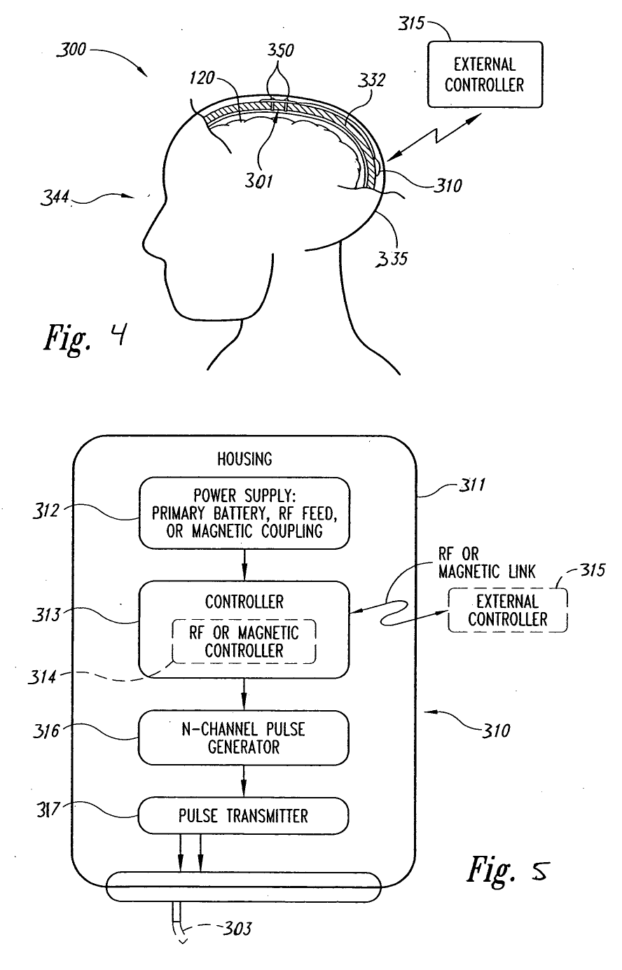

The pulse system 310 can be implanted at a subclavicular location, as shown in FIG. 3. In particular embodiments, the pulse system 310 (and/or other implanted components of the system 300) can include titanium and/or other materials that can be exposed to magnetic fields generated by magnetic resonance chambers without harming the patient. The pulse system 310 can also be controlled internally via pre-programmed instructions that allow the pulse system 310 to operate autonomously after implantation. In other embodiments, the pulse system 310 can be implanted at other locations, and at least some aspects of the pulse system 310 can be controlled externally. For example, FIG. 4 illustrates an embodiment of the system 300 in which the pulse system 310 is positioned on the external surface of the skull 332, beneath the scalp 335. The pulse system 310 can be controlled internally and/or via an external controller 315.

FIG. 5 schematically illustrates a representative example of a pulse system 310 suitable for use in the neural stimulation system 300 described above. The pulse system 310 generally includes a housing 311 carrying a power supply 312, an integrated controller 313, a pulse generator 316, and a pulse transmitter 313. The power supply 312 can be a primary battery, such as a rechargeable battery or other suitable device for storing electrical energy. In other embodiments, the power supply 312 can be an RF transducer or a magnetic transducer that receives broadcast energy emitted from an external power source and that converts the broadcast energy into power for the electrical components of the pulse system 310.

In one embodiment, the integrated controller 313 can include a processor, a memory, and a programmable computer medium. The integrated controller 313, for example, can be a microcomputer, and the programmable computer medium can include software loaded into the memory of the computer, and/or hardware that performs the requisite control functions. In another embodiment identified by dashed lines in FIG. 5, the integrated controller 313 can include an integrated RF or magnetic controller 314 that communicates with the external controller 315 via an RF or magnetic link. In such an embodiment, many of the functions performed by the integrated controller 313 may be resident on the external controller 315 and the integrated portion 314 of the integrated controller 313 may include a wireless communication system.

The integrated controller 313 is operatively coupled to, and provides control signals to, the pulse generator 316, which may include a plurality of channels that send appropriate electrical pulses to the pulse transmitter 317. The pulse generator 316 may have multiple channels, with at least one channel associated with a particular one of the electrodes 350 described above. The pulse generator 316 sends appropriate electrical pulses to the pulse transmitter 317, which is coupled to the electrodes 350 (FIG. 1). In one embodiment, each of these electrodes 350 is configured to be physically connected to a separate lead, allowing each electrode 350 to communicate with the pulse generator 316 via a dedicated channel. Suitable components for the power supply 312, the integrated controller 313, the external controller 315, the pulse generator 316, and the pulse transmitter 317 are known to persons skilled in the art of implantable medical devices.

The pulse system 310 can be programmed and operated to adjust a wide variety of stimulation parameters, for example, which electrodes are active and inactive, whether electrical stimulation is provided in a unipolar or bipolar manner, and/or how the stimulation signals are varied. In particular embodiments, the pulse system 310 can be used to control the polarity, frequency, duty cycle, amplitude, and/or spatial and/or temporal qualities of the stimulation. The stimulation can be varied to match naturally occurring burst patterns (e.g., theta burst stimulation), and/or the stimulation can be varied in a predetermined, pseudorandom, and/or aperiodic manner at one or more times and/or locations.

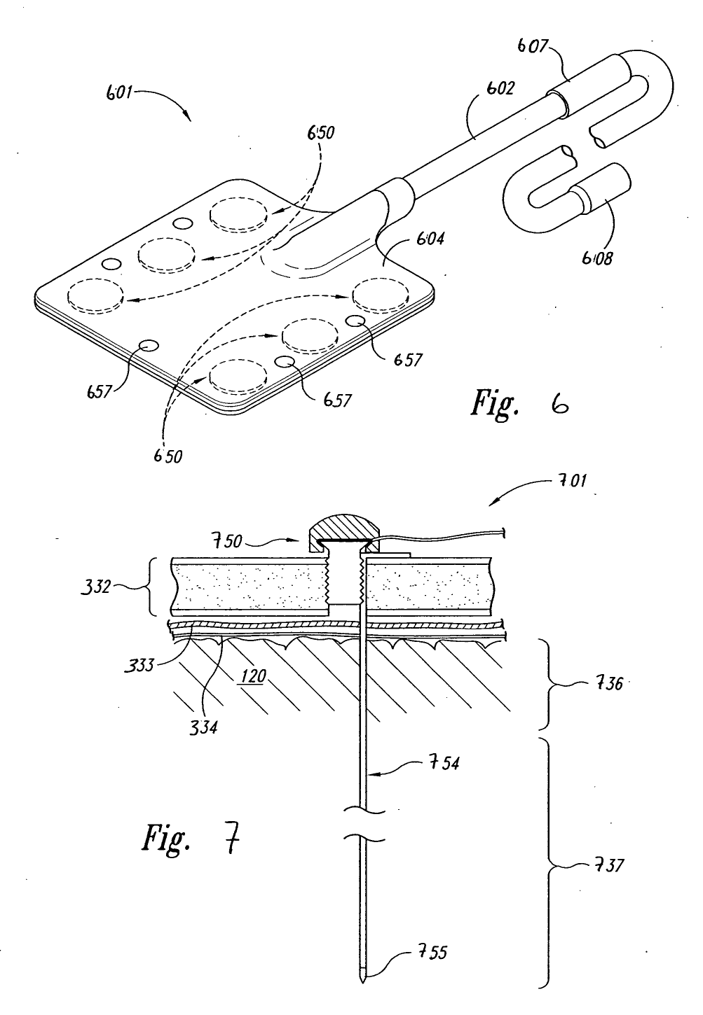

Stimulation can be provided to the patient using devices in addition to or in lieu of those described above. For example, FIG. 6 is a top, partially hidden isometric view of an embodiment of an electrode device 601 configured to carry multiple cortical electrodes 650. The electrodes 650 can be carried by a flexible support member 604 (located within the patient's skull) to place each electrode 650 at a stimulation site of the patient when the support member 604 is implanted within the patient's skull. Electrical signals can be transmitted to the electrodes 650 via leads carried in a communication link 603. The communication link 603 can include a cable 602 that is connected to the pulse system 310 (FIG. 3) via a connector 608, and is protected with a protective sleeve 607. Coupling apertures or holes 657 can facilitate temporary attachment of the electrode device 601 to the dura mater at, or at least proximate to, a stimulation site. The electrodes 650 can be biased cathodally and/or anodally, as described above. In an embodiment shown in FIG. 6, the electrode device 601 can include six electrodes 650 arranged in a 2×3 electrode array (i.e., two rows of three electrodes each), and in other embodiments, the electrode device 601 can include more or fewer electrodes 650 arranged in symmetrical or asymmetrical arrays. The particular arrangement of electrodes 650 can be selected based on the region of the patient's brain that is to be stimulated, and/or the patient's condition.

In a particular embodiment, a device generally similar to the device shown in FIG. 6 can be constructed and positioned to extend over both the first neural population 130 (FIG. 2) and the second neural population 131 (FIG. 2). Accordingly, the practitioner can implant a single device that allows the practitioner to stimulate motor neurons (or another neural population used to determine stimulation parameters) and provide stimulation to a population of silent neurons (e.g., cognitive neurons or other silent neurons). The stimulation of motor neurons and silent neurons may occur simultaneously, sequentially, or separately. The electrode device may include a two-dimensional array of electrodes as shown in FIG. 6, or can include a linear arrangement or other arrangement of electrodes, depending upon the particular neural populations to be stimulated.

FIG. 7 illustrates an electrode device 701 that may be configured to apply electrical stimulation signals to a cortical region 736 or a subcortical region 737 of the brain 120 in accordance with further embodiments of the invention. The electrode device 701 can include an electrode 750 having a head and a threaded shaft that extends through a pilot hole in the patient's skull 332. If the electrode 750 is intended for cortical stimulation, it can extend through the skull 332 to contact the dura mater 333 or the pia mater 334. If the electrode 750 is to be used for subcortical stimulation, it can include an elongate conductive member 754 that extends downwardly through the cortical region 736 into the subcortical region 737. Most of the length of the elongate conductive member 754 can be insulated, with just a tip 755 exposed to provide electrical stimulation in only the subcortical region 737. Subcortical stimulation may be appropriate in at least in some instances, for example, when the brain structures such as the basal ganglia are to be stimulated. In other embodiments, other deep brain structures (e.g., the amygdala or the hippocampus) can be stimulated using a subcortical electrode. If the hippocampus is to be stimulated, stimulation may be provided to the perihippocampal cortex using a subdurally implanted electrode that need not penetrate through brain structures other than the dura.

Further details of electrode devices that may be suitable for electromagnetic stimulation in accordance with other embodiments of the invention are described in the following pending U.S. Patent Applications, all of which are incorporated herein by reference: 10/891,834, filed Jul. 15, 2004; Ser. No. 10/418,796, filed Apr. 18, 2003; and Ser. No. 09/802,898, filed Mar. 8, 2001. Further devices and related methods are described in a copending U.S. Application No. ______, titled “Systems and Methods for Patient Interactive Neural Stimulation and/or Chemical Substance Delivery,” (Attorney Docket No. 33734.8082US) and U.S. Application No. ______, titled “Methods and Systems for Establishing Parameters for Neural Stimulation,” (Attorney Docket No. 33734.8079US), both filed concurrently herewith and incorporated herein by reference.

In still further embodiments, other techniques may be used to provide stimulation to the patient's brain. Such techniques can include electromagnetic techniques in addition to purely electrical techniques. In particular, such techniques can include transcranial magnetic stimulation techniques, which do not require that an electrode be implanted beneath the patient's skull. In still further embodiments, other techniques, which also may not require an implant, can be used. Such additional techniques can include transcranial direct current stimulation.

D. Techniques For Detecting a Response to Neural Stimulation

Once the appropriate stimulation device has been selected and positioned, the practitioner can apply stimulation and, particularly if the practitioner is stimulating the first neural population, detect a response. The practitioner may also wish to detect a response when stimulation is applied to the second neural population, e.g., to verify that the stimulation provided in accordance with the second set of stimulation parameters is or appears to be producing a desired response, condition, state, or change. In a particular aspect of either process, the response is detected at least proximate to the patient's central nervous system, and in a further particular aspect, at the patient's brain. One or more of several techniques may be employed to determine the neural response to the stimulation. Many suitable techniques rely on hemodynamic properties, e.g., they measure or are based on concentrations of oxy-hemoglobin and/or deoxy-hemoglobin. Such techniques can include functional magnetic resonance imaging (fMRl), measurements or estimates of cerebral blood flow, cerebral blood volume, cerebral metabolic rate of oxygen (CMRO), Doppler flowmetry, and/or optical spectroscopy using near infrared radiation. Magnetic resonance techniques (e.g., fMRI techniques) can be performed inside a magnetic resonance chamber, as described below with reference to FIG. 8.

Certain other techniques, e.g., thermal measurements and/or flowmetry techniques, can be performed subdermally on the patient. Still further techniques, in particular, optical techniques such as near infrared spectroscopy techniques, are generally noninvasive and do not require penetration of the patience's scalp or skull. These techniques can include placing a near infrared emitter and detector (or an array of emitter/detector pairs) on the patient's scalp to determine species concentrations of both oxy-hemoglobin and deoxy-hemoglobin. Representative devices for measuring hemodynamic quantities (that correspond to neural activity) are disclosed in U.S. Pat. No. 5,024,226, U.S. Pat. No. 6,615,065, both incorporated herein by reference, and are available from ISS, Inc. of Champaign, Ill., and Somanetics of Troy, Mich. Further devices and associated methods are disclosed in pending U.S. Application No. ______, titled “Neural Stimulation and Optical Monitoring Systems and Methods,” (Attorney Docket No. 33734-8084US), filed concurrently herewith and incorporated herein by reference. Any of the foregoing techniques can be used to identify and/or quantify parameters and/or states associated with the patient's level of neural functioning. Such states may determine, influence, and/or alter signal properties such as intensity, power, spectral, phase, coherence, and/or other signal characteristics.

FIG. 8 illustrates a magnetic resonance imaging system 840 having a patient platform 841 for carrying the patient during a procedure for detecting responses to stimulation. Functional MRI techniques can be used to correlate levels of brain activity with stimulation provided to the patient's brain via one or more stimulation parameters. If the stimulation is to be provided via implanted devices, the implanted devices are selected to be compatible with the strong magnetic fields generated by the chamber.

Some embodiments of the invention may involve magnetic resonance spectroscopy (MRS) techniques, which may facilitate the identification or determination of various chemical species and/or relative concentration relationships between such species in particular brain regions. Stimulation sites may be selected based upon, for example, a detected imbalance between particular neurotransmitters. Additionally or alternatively, the effect(s) of neural stimulation may be evaluated or monitored on a generally immediate, short term, and/or long term basis using MRS and/or other imaging techniques.

FIG. 9 illustrates a patient wearing an electrode net 943 that includes a network of receptor electrodes positioned over the patient's scalp to sense, detect, or measure electroencephalographic (EEG) signals corresponding to the patient's neuroelectric activity. In a representative embodiment, the electrode net 943 may include a Geodesic Sensor Net manufactured by Electrical Geodesics, Inc., of Eugene, Oreg. When external or non-intrinsic electromagnetic stimulation generates or affects a locational, spectral, and/or temporal response or change in the patient's neuroelectric activity, such responses or changes in the patient's neuroelectric signals can be sensed or detected by the electrode net 943. Accordingly, the detected properties of or changes in neuroelectric signals (or the relative absence of particular characteristics or changes) can be used to determine whether the threshold level for a target neural population has been met. In particular embodiments, the foregoing sensors can provide coherence information, which relates to the rhythmic or synchronous aspects of the patient's neural activity. Further details regarding coherence are disclosed in co-pending U.S. application Ser. No. 10/782,526, filed on Feb. 19, 2004 and incorporated herein by reference.

In other embodiments, a net (or other network) generally similar to that shown in FIG. 9 can be outfitted with sensors other than electrical sensors. For example, such a net can be outfitted with near infrared sensors or other optical sensors. Such sensors may detect changes in neural activity arising in association with subthreshold, threshold, and/or suprathreshold level electromagnetic stimulation.

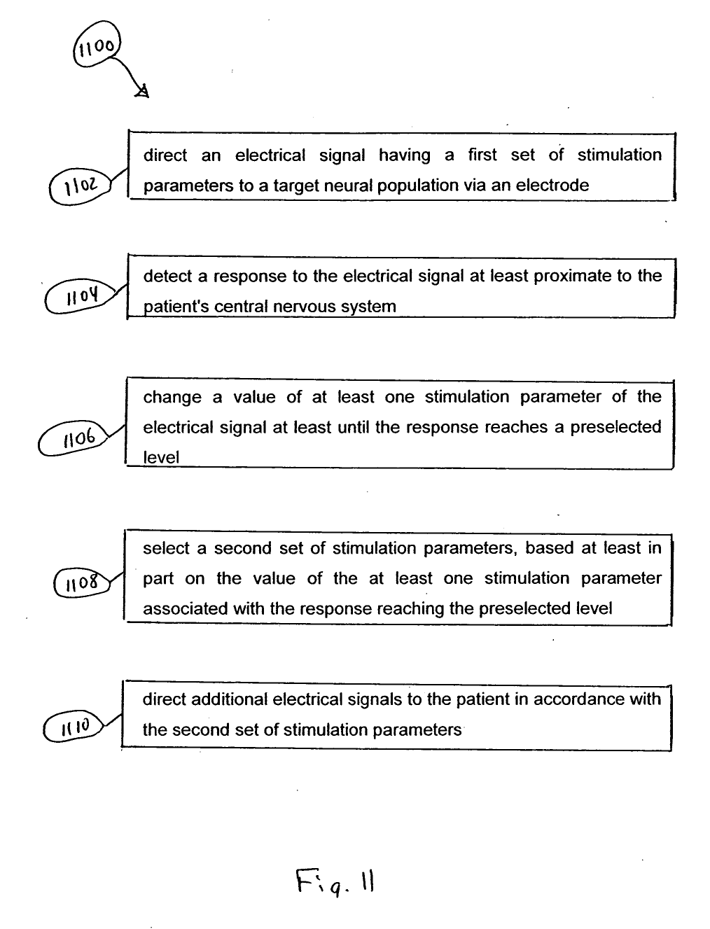

The method described above with reference to FIG. 1C is directed generally to using responses obtained from stimulating a first neural population to determine stimulation parameters for stimulating a second (functionally different) neural population. FIG. 10 is a flow diagram illustrating a more specific application of such a method. The process 1000 shown in FIG. 10 can include at least estimating a stimulation parameter for a motor neural population (process portion 1002). This can include stimulating the motor neural population (process portion 1004) detecting a first patient response resulting from the stimulation (process portion 1006), and detecting a second patient response, also resulting from stimulating the motor neural population (process portion 1008). The second patient response can be of a type that results from stimulating both motor neurons and non-motor neurons. Based at least in part on the second patient response, the method can further include at least estimating (in particular embodiments, determining and/or selecting) a stimulation parameter for a non-motor neural population (process portion 1010).

In a particular application of the process 1000, stimulating the motor neural population can include applying electrical stimulation to a neural population located at the primary motor cortex. Detecting a first patient response resulting from stimulating the motor neural population can include detecting evidence that the stimulation has met or exceeded the level required for activation of the neural population. For example, detecting the first patient response can include observing or measuring a muscle action by the patient. Detecting the second patient response can include detecting a physiological characteristic that is shared by the first and second neural populations, for example, detecting a change in cerebral blood flow or other hemodynamic quantity, or detecting an electrical signal emitted by the motor neural population. The second patient response can be generally simultaneous with the first patient response (or at least clearly linked with the first patient response). For example, if it is determined that the cerebral blood flow changes by a certain amount (or has a certain value) when the motor neuron is stimulated at a current and/or voltage sufficient to produce an action potential, this information can be used to provide similar stimulation to the non-motor neural population. Accordingly, the non-motor neural population may not exhibit a response similar to the first patient response, but may exhibit the second patient response. By correlating the second patient response with the first patient response using the motor neural population, the non-motor neural population can be stimulated in a manner at least correlated with (and in some cases, generally similar to) that of the motor neural population, without requiring the non-motor neural population to exhibit the first patient response (e.g., the muscle action). In other embodiments, a generally similar approach can be followed, using different neurons to generate the first patient response. For example, sensory neurons can be stimulated to generate a first patient response that includes a sensation by the patient. The second patient response can be generally the same as any of those described above (e.g., a hemodynamic response).

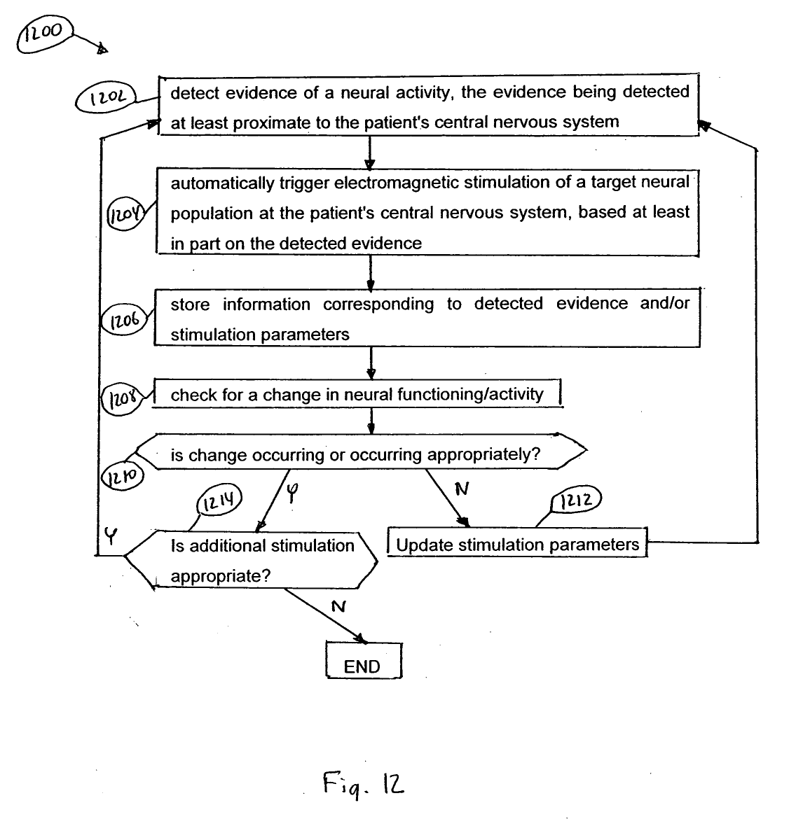

FIG. 11 is a flow diagram illustrating a method 1100 for treating a patient in accordance with another embodiment of the invention. The method 1100 can include directing an electrical signal having a first set of stimulation parameters to a target neural population via an electrode (process portion 1102). The method can further include detecting a response to the electrical signal at least proximate to the patient's central nervous system (process portion 1104). For example, process portion 1104 can include detecting a hemodynamic response, electrical response, or other response at the patient's brain or other portion of the patient's central nervous system. In process portion 1106, a value of at least one stimulation parameter of the electrical signal is changed at least until the response reaches a preselected level. For example, a spatial, temporal, and/or waveform (e.g., polarity, current, voltage, pulse width, or pulse repetition frequency) parameter of the electrical signal can be varied to achieve a preselected response level. The response level can correspond to a threshold level in some embodiments, and in other embodiments, can correspond to a subthreshold level or a suprathreshold level. In process portion 1108, a second set of stimulation parameters is selected, based at least in part on the value of the at least one stimulation parameter associated with (e.g., occurring at the same time as) the response reaching the preselected level. Accordingly, the response to the electrical signal provided with the first set of stimulation parameters can influence the choice of a second set of stimulation parameters, which is then used to direct additional signals to the patient (process portion 1110). The additional signals can be directed to the same target neural population, and/or to a different neural population.

The technique described above with reference to FIG. 11 used to determine stimulation parameters for non-motor, non-sensory and/or other silent neurons, and in certain embodiments, parameters for motor and/or sensory neurons as well. For example, the preselected level can be determined based on stimulation levels obtained from motor or sensory neurons, (as described above with reference to FIG. 10), or can be based upon data indicating improved functionality at that preselected level for other similarly situated patients. Accordingly, the preselected level need not be obtained from motor or sensory data. In another embodiment, the foregoing method may also be applied to motor or sensory neurons during the course of therapies directed at treating such neurons, without the need for monitoring an externally exhibited patient response when a threshold simulation level is achieved. Instead, a practitioner can refer to existing data corresponding to the selected level, or can identify a level, transition, shift, “jump” or other change in a parameter that is correlated with a desired change in patient functionality. For example, the practitioner can observe a change in a hemodynamic quantity that, for a particular patient, or over a multi-patient population, has been associated with patient improvement and is therefore appropriate as a stimulation parameter.

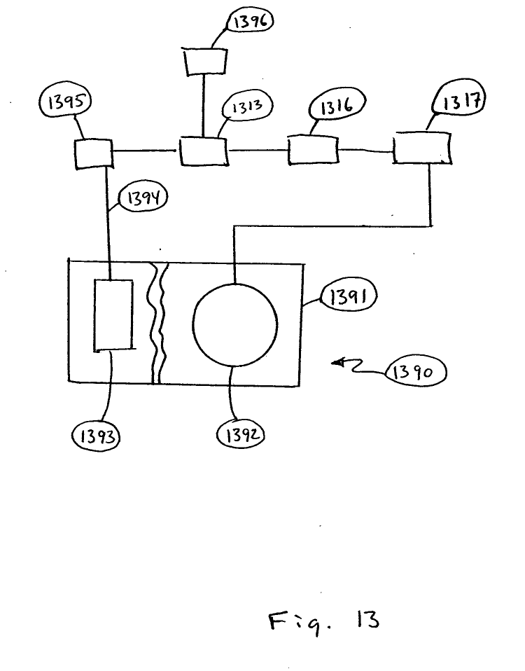

FIG. 12 is a flow diagram illustrating a process 1200 for providing electromagnetic stimulation to a patient. Process portion 1202 can include detecting evidence of a neural activity, with the evidence being detected at least proximate to the patient's central nervous system. In process portion 1204, electromagnetic stimulation of a target neural population at least proximate to the patient's central nervous system is automatically triggered, adjusted, interrupted, resumed, or discontinued, based at least in part on the detected evidence. For example, any of the foregoing techniques relating to hemodynamic properties and/or neuroelectric properties (e.g., EEG or electrocorticographic (ECoG) signals) can provide evidence of a neural activity, and once the neural activity is detected, electromagnetic stimulation can automatically be triggered, adjusted, interrupted, resumed or discontinued. In certain cases, triggering or adjusting electrical stimulation may aid patients whose level of neural functioning is such that at least some neural activity is generated by the patient when the patient undertakes or attempts to undertake a neural task. When such neural activity is detected, the automatically generated electromagnetic stimulation may be provided at a level that affects neural membrane potentials in a manner that at least makes the generation of action potentials by a target neural population more likely, such that weak or relatively weak intrinsic neural signals have a greater chance of triggering a corresponding neural function, thereby subserving neurofunctional development (e.g., by one or more biological mechanisms associated with neuroplasticity). The automatically generated electromagnetic stimulation may result in an immediate and/or long lasting improved level of neural functioning. Because the process of providing the stimulation is automated, neither the patient nor a practitioner need take any action beyond the patient generating some level(s) of neural activity. In particular embodiments, an initial level of neural activity can correspond to the patient's attempt to engage in a physical or cognitive activity. While the patient's mere attempt may not by itself be enough to generate the desired movement or cognition, the attempt in combination with the automatically triggered stimuli is expected to be enough to do so.

In further particular embodiments, the process 1200 can include storing information corresponding to the detected evidence and/or the stimulation levels (process portion 1206). This information can be used by the practitioner to track parameters associated with the stimulation (e.g., how often the stimulation is triggered, and what characteristics the stimulation signals have). The process can also include checking for a change in neural function and/or activity (process portion 1208). In process portion 1210, it can be determined whether the change is occurring, or if it is occurring, whether it is occurring appropriately (e.g., at the appropriate pace and/or in the appropriate direction). If not, the stimulation parameters can be updated (process portion 1212) and the method can return to process portion 1202. In a particular embodiment, this feedback process can be used to identify changes or drifts in the patient's threshold stimulation levels over the course of a treatment regimen, and can automatically update the stimulation parameters accordingly. If the change is occurring appropriately, the process can further include checking to see if additional stimulation (with the existing stimulation parameters) is appropriate (process portion 1214). If so, the process returns to process portion 1202. If not, the process can end.

In at least some embodiments, process portion 1202 can include detecting hemodynamic properties that tend to change in response to changes in the patient's neural activity level(s). In many cases, an increase in perfusion levels can indicate a (desirable) increase in brain activity levels. However, this is not always the case. For example, some neuropsychiatric disorders (e.g., attention deficit disorder) can be accompanied by hyperperfusion in particular brain areas. Conversely, other neuropsychiatric disorders (e.g. depression) and some types of neuropsychiatric or cognitive dysfunctions may be indicated by hypoperfusion of a target neural area, and in still other disorders, a patient's brain may exhibit hypoperfusion in certain neural regions and hyperperfusion in other neural regions. Accordingly, effective therapy may be detected by noting or detecting a desirable or undesirable perfusion condition in one or more target neural populations. Effective treatment (e.g., provided by electrical stimulation, possibly in association with an adjunctive therapy such as behavioral therapy and/or drug therapy) may shift perfusion levels in particular target neural populations toward more normal or desirable levels. In some cases, the foregoing effects may be hidden or partially hidden by medications the patient takes, because such medications may directly or indirectly affect a neural population under consideration. Accordingly, one technique for detecting evidence of neural activity can include performing a check on a neural activity level after the patient has ceased taking a drug, as the effects of the drug wear off, and/or after the drug has worn off and the patient has returned to a “drug-off” state.

In some cases detecting evidence of neural activity can include detecting a particular value of a parameter (e.g., blood flow volume or oxygen content) that corresponds to an activity level. In other embodiments, detection includes detecting a change, rather than a particular value, of the parameter. The nature of these changes may be specific to individual patients, and/or may vary with the patient's condition. For example, changes may be quantitatively and/or qualitatively different for patients of different ages.

FIG. 13 is a schematic illustration of an implantable stimulation and monitoring interface 1390 configured for stimulating a target neural population and detecting signals corresponding to neural activity according to an embodiment of the invention. Accordingly, embodiments of the interface 1390 can be used to carry out the process 1200 described above with reference to FIG. 12. Some or all aspects of the interface 1390 shown in FIG. 13 can be incorporated into any of the devices described above with reference to FIGS. 3-7. In one embodiment, the stimulation and monitoring interface 1390 comprises a support member 1391 carrying at least one stimulating element 1392 and at least one monitoring element 1393. The stimulating element 1392 may include one or more electrodes organized in accordance with a particular pattern, and the monitoring element 1393 may include a set of electrodes and/or a monitoring device positioned proximate or adjacent to the stimulating element 1392. In a particular embodiment, the stimulating element 1392 and the monitoring element 1393 can have a fixed relationship to each other. Accordingly, the interface 1390 can stimulate and monitor the same neural population, or stimulate one neural population and detect a response at another neural population spaced apart by the fixed distance. In another embodiment, these elements can be separate from or movable relative to each other (e.g., carried by different structures or support members), as indicated by broken lines, so that the practitioner has greater flexibility in selecting a set of neural populations for stimulation and one or more other neural populations for response detection. In a further aspect of this embodiment, one element (e.g., the stimulating element 1392) can be implanted to stimulate a particular neural population, and the other element (e.g., the monitoring element 1393) can be located external to the patient (e.g., at the patient's scalp) to monitor the same or a different neural population.

A lead or link 1394 may couple the monitoring element 1393 to a sensing unit 1395. The sensing unit 1395 may in turn be coupled to a controller 1313, pulse generator 1316, and pulse transmitter 1317, which are coupled back to the stimulating element 1393. Accordingly, the monitoring element 1393 can detect signals indicative of neural activity associated with particular neural populations and, via the controller 1313, can direct the stimulating element 1392 to deliver or apply stimulation signals to the same or a different target neural population. Information corresponding to the sensed data and/or the stimulation data can be stored at a memory device 1396 or other computer-readable medium (e.g., an implanted memory and/or external memory or disk drive). Aspects of some or all of the foregoing functionalities can reside on programmable computer-readable media.

In a particular embodiment, the monitoring element 1393 may include an array of cortical sensing electrodes, a deep brain electrode, and/or one or more other electrode types. In other embodiments, the monitoring element can include devices generally similar to those described above for monitoring hemodynamic quantities (e.g., optical spectroscopy monitors, cerebral blood flow monitors, cerebral blood volume monitors, Doppler flowmetry monitors, and/or others).

In some embodiments (e.g., when the monitoring element monitors electrical signals), the delivery of stimulation signals to a target neural population may interfere with the detection of signals corresponding to neural activity. As a result, the controller 1313 and/or the pulse system 1316 may periodically interrupt a neural stimulation procedure, such that during stimulation procedure interruptions, the sensing unit 1395 may analyze signals received from the monitoring element 1393. Outside of such interruptions, the sensing unit 1395 may be prevented from receiving or processing signals received from the monitoring element 1393. In particular embodiments, stimulation pulses may be interleaved with sensing “windows” so that the stimulation and monitoring tasks may be performed in alternating succession. In other embodiments, the sensing unit 1395 may compensate for the presence of stimulation signals, for example, through signal subtraction, signal filtering, and/or other compensation operations, to facilitate detection of neural activity or evidence of neural activity simultaneous with the delivery of stimulation signals to a target neural population.

In embodiments in which a neural stimulation procedure is periodically interrupted to facilitate detection of neural activity or evidence of such activity, the interface 1390 may include a single electrode arrangement or configuration in which any given electrode element used to deliver stimulation signals during the neural stimulation procedure may also be used to detect neural activity during a neural stimulation procedure interruption.

From the foregoing, it will be appreciated that specific embodiments of the invention have been described herein for purposes of illustration, but that various modifications may be made without deviating from the invention. For example, in some embodiments, data obtained from a first neural population can be used to identify stimulation parameters for a second neural population of the same patient. In other embodiments, data obtained from stimulating one type of neural population in one patient can be used to at least influence the choice of stimulation parameters selected for a different type of neural population in a different patient. Once stimulating parameters for a particular target neural population have been identified, a corresponding treatment regimen can include adjunctive therapies in addition to electromagnetic stimulation. Adjunctive therapies can include cognitive-based activities when the target neural population includes neurons associated with such activities, and/or other types of activities (e.g., physical therapy, auditory activities, visual tasks, speech production or language comprehension) for neurons associated therewith. Adjunctive therapies can also include drug-based therapies. Aspects of the invention described in the context of particular embodiments may be combined or eliminated in other embodiments. For example, aspects of the automated feedback system described in the context of FIG. 13 may be combined with aspects of the stimulation devices described with reference to FIGS. 3-7. While advantages associated with certain embodiments of the invention have been described in the context of those embodiments, other embodiments may also exhibit such advantages, and not all embodiments need necessarily exhibit such advantages to fall within the scope of the invention. Accordingly, the invention is not limited except as by the appended claims.

I/We claim:

1. A method for treating a patient, comprising:
applying a first stimulus to a first neural population associated with a first neural function, using a first set of stimulation parameters;
detecting a response to the first stimulus at least proximate to the patient's central nervous system; and
based at least in part on the response to the first stimulus and on the first set of stimulation parameters, applying a second stimulus to a second neural population associated with a second neural function different than the first neural function using a second set of stimulation parameters.

2. The method of claim 1 wherein detecting a response to the first stimulus includes detecting a response that is also exhibited by the second neural population.

3. The method of claim 1 wherein using a second set of stimulation parameters includes using a second set of parameters that differs from the first set of parameters.

4. The method of claim 1 wherein using a second set of stimulation parameters includes using a second set of parameters that is at least approximately the same as the first set of parameters.

5. The method of claim 1 wherein applying the first stimulus includes applying a first electrical signal and wherein applying the second stimulus includes applying a second electrical signal.

6. The method of claim 1 wherein detecting a response includes detecting an electrical signal transmitted by the central nervous system.

7. The method of claim 1 wherein detecting a response includes detecting a hemodynamic quantity.

8. The method of claim 1 wherein detecting a response includes detecting a change in cerebral blood flow.

9. The method of claim 1 wherein detecting a response includes detecting a change in a quantity that depends upon cerebral blood flow.

10. The method of claim 1 wherein detecting a response includes detecting a change in a quantity that depends upon cerebral blood oxygen levels.

11. The method of claim 1 wherein applying a first stimulus to a first neural population includes applying a first stimulus to a first neural population associated with a sensory function.

12. The method of claim 1 wherein applying a first stimulus to a first neural population includes applying a first stimulus to a first neural population associated with a motor function.

13. The method of claim 1 wherein applying a second stimulus to a second neural population includes applying a second stimulus to a second neural population associated with a neuropsychological function.

14. The method of claim 1 wherein applying a second stimulus to a second neural population includes applying a second stimulus to a second neural population associated with a cognitive function.

15. A method for treating a patient, comprising:
directing an electrical signal having a first set of stimulation parameters to a target neural population via an electrode;
detecting a response to the electrical signal at least proximate to the patient's central nervous system;
changing a value of at least one stimulation parameter of the electrical signal at least until the response reaches a preselected level;
selecting a second set of stimulation parameters, based at least in part on the value of the at least one stimulation parameter associated with the response reaching the preselected level; and
directing additional electrical signals to the patient in accordance with the second set of stimulation parameters.

16. The method of claim 15 wherein directing additional signals includes directing additional signals to the target neural population.

17. The method of claim 15 wherein the target neural population is a first target neural population, and wherein directing additional signals includes directing additional signals to a second target neural population different than the first target neural population.

18. The method of claim 15 wherein changing a value of at least one stimulation parameter includes changing a value of at least one of a voltage and current with which the electrical signal is applied.

19. The method of claim 15 wherein changing a value of at least one stimulation parameter includes changing a value of at least one of aspect of a waveform with which the electrical signal is applied.

20. The method of claim 15 wherein the preselected level corresponds at least approximately to a threshold stimulation level of the target neural population.

21. The method of claim 15 wherein detecting a response includes detecting a response that evidences a neural activity level.

22. The method of claim 21 wherein detecting a response includes detecting a characteristic that depends on a cerebral blood oxygen level.

23. A method for evaluating neural functioning in a patient, comprising:
at least estimating a stimulation parameter for a motor neural population by:
stimulating the motor neural population;
detecting a first patient response resulting from stimulating the motor neural population;
detecting a second patient response resulting from stimulating the motor neural population, the second patient response being of a type that results from stimulating both motor neurons and non-motor neurons; and
based at least in part on the second patient response, at least estimating a stimulation parameter for a non-motor neural population.

24. The method of claim 23 wherein stimulating the motor neural population includes applying an electrical signal to the motor neural population.

25. The method of claim 23 wherein at least estimating a stimulation parameter for a motor neural population includes determining a stimulation parameter.

26. The method of claim 23 wherein detecting a first patient response includes detecting a response that the non-motor neurons do not produce.

27. The method of claim 23 wherein detecting the first patient response includes detecting a motor response.

28. The method of claim 23 wherein detecting the first patient response includes detecting an improvement in a neurologically-based symptom.

29. The method of claim 23 wherein detecting a second patient response includes detecting a response evidencing neural activity.

30. The method of claim 29 wherein detecting a second patient response includes detecting a change in hemodynamic quantity.

31. The method of claim 23 wherein at least estimating a stimulation parameter for a non-motor neural population includes at least estimating a level of electrical current to be applied to the non-motor neural population.

32. The method of claim 23 wherein at least estimating a stimulation parameter for a non-motor neural population includes at least estimating a waveform of an electrical signal to be applied to the non-motor neural population.

33. The method of claim 23 wherein detecting a non-motor response includes detecting a non-motor response that is at least approximately concurrent with the motor response.

34. The method of claim 23 wherein the non-motor response is a first non-motor response, and wherein the method further comprises:
applying an electromagnetic stimulation signal to the non-motor neural population;
detecting a second non-motor response from the non-motor neural population, the second non-motor response being at least similar in type to the first non-motor response; and
selecting a characteristic of the electromagnetic stimulation signal applied to the non-motor neural population to produce a level of the second non-motor response that is approximately the same as a corresponding level of the first non-motor response.

35. The method of claim 23 wherein detecting a non-motor response includes detecting an electrical signal emitted by the motor neural population.

36. The method of claim 23 wherein detecting a non-motor response includes detecting a change using near infrared imaging techniques.

37. The method of claim 23, further comprising stimulating the non-motor neural population.

38. The method of claim 23, further comprising stimulating the non-motor neural population at sub-threshold levels.

39. A method for evaluating neural functioning in a patient, comprising:
determining a threshold electromagnetic stimulation parameter for a motor neural population by:
applying an electromagnetic stimulation signal to the motor neural population;
detecting a motor response resulting from stimulating the motor neural population; and
detecting a first non-motor response resulting from stimulating the motor neural population;
applying an electromagnetic stimulation signal to a non-motor neural population;
detecting a second non-motor response from the non-motor neural population, the second non-motor response being at least similar in type to the first non-motor response; and
selecting a characteristic of the electromagnetic stimulation signal applied to the non-motor neural population to produce a level of the second non-motor response that is approximately the same as a corresponding level of the first non-motor response.

40. The method of claim 39 wherein detecting a first non-motor response includes detecting a first non-motor response that is at least approximately concurrent with the motor response.

41. The method of claim 39 wherein detecting a motor response resulting from stimulating the motor neural population includes detecting a motor response resulting from threshold or suprathreshold stimulation, and wherein the method further comprises:
detecting a change in the threshold level for the motor neural population;
changing the characteristic of the electromagnetic stimulation signal applied to the non-motor neural population based at least in part on the detected change in the threshold level for the motor neural population.

42. A method for evaluating neural functioning in a patient, comprising:
at least estimating a stimulation parameter for a sensory neural population by:
stimulating the sensory neural population;
detecting a first patient response resulting from stimulating the sensory neural population;
detecting a second patient response resulting from stimulating the sensory neural population, the second patient response being of a type that results from stimulating both sensory neurons and non-sensory neurons; and
based at least in part on the second patient response, at least estimating a stimulation parameter for a non-sensory neural population.

43. The method of claim 42 wherein stimulating the sensory neural population includes applying an electrical signal to the motor neural population.

44. The method of claim 42 wherein detecting a first patient response includes detecting a response that the non-sensory neurons do not produce.

45. The method of claim 42 wherein at least estimating a stimulation parameter for a non-sensory neural population includes at least estimating a level of electrical current to be applied to the non-sensory neural population.

46. The method of claim 42 wherein at least estimating a stimulation parameter for a non-sensory neural population includes at least estimating a waveform of an electrical signal to be applied to the non-sensory neural population.

47. The method of claim 42 wherein detecting a non-sensory response includes detecting a non-sensory response that is at least approximately concurrent with the sensory response.

48. A method for treating a patient, comprising:
detecting evidence of a neural activity, the evidence being detected at least proximate to the patient's central nervous system; and
automatically triggering electromagnetic stimulation of a target neural population at the patient's central nervous system, based at least in part on the detected evidence.

49. The method of claim 48, further comprising automatically adjusting at least one parameter with which stimulation is applied to the patient, based at least in part on the detected evidence of neural activity.

50. The method of claim 48, further comprising automatically saving data computer-readable medium data corresponding to the evidence of neural activity, or the electromagnetic stimulation, or both.

51. The method of claim 48 wherein detecting evidence of a neural activity includes detecting evidence of a neural activity exhibited by a first neural population, and wherein stimulating a target neural population includes stimulating a second neural population different than the first neural population.

52. The method of claim 48 wherein detecting evidence of a neural activity includes detecting evidence of a neural activity exhibited by the target neural population.

53. The method of claim 48 wherein detecting evidence of a neural activity includes detecting evidence of the patient's attempt to engage in a neural activity.

54. The method of claim 58 wherein detecting evidence of a neural activity includes detecting evidence of the patient's attempt to engage in a cognitive activity.

55. The method of claim 48 wherein detecting evidence of a neural activity includes detecting evidence of a neuropsychiatric activity.

56. The method of claim 48 wherein detecting evidence of a neural activity includes detecting a cerebral blood oxygen level.

57. The method of claim 56 wherein detecting evidence of a neural activity includes detecting a decrease in cerebral blood oxygen level.

58. The method of claim 56 wherein detecting evidence of a neural activity includes detecting an increase in cerebral blood oxygen level.

59. The method of claim 48 wherein detecting evidence of a neural activity includes detecting a change in a parameter evidencing neural activity.

60. The method of claim 59 wherein detecting evidence of a neural activity includes detecting a change in a parameter evidencing neural activity after the patient has changed a medicament intake level.

61. A neurostimulation apparatus, comprising:
a sensor configured to be placed at least proximate to a patient's central nervous system, the sensor being configured to detect evidence of neural activity and transmit a response signal;
a stimulation device configured to be placed at least proximate the patient's central nervous system and direct a stimulation signal; and
a controller operatively coupled to the sensor and the stimulation device, the controller having instructions for automatically directing the stimulation device to direct the stimulation signal, based at least in part on the response signal received from the sensor.

62. The apparatus of claim 61 wherein the controller includes instructions for automatically changing stimulation parameters in accordance with which the stimulation signal is directed, based at least in part on the response signal received from the sensor.

63. The apparatus of claim 61, further comprising a computer readable storage medium operatively coupled to the controller to automatically store data corresponding to the evidence of neural activity, or the electromagnetic stimulation, or both.

64. The apparatus of claim 61 wherein the sensor and the stimulation device are positioned adjacent to each other and have a fixed relationship to each other.

65. The apparatus of claim 61 wherein the sensor and the stimulation device are movable relative to each other.

66. The apparatus of claim 61 wherein the controller includes a computer-readable medium having the instructions for directing the stimulation device.

67. The apparatus of claim 66 wherein the computer-readable medium is programmable.

68. The apparatus of claim 61 wherein the stimulation device includes an implantable electrode.

69. The apparatus of claim 61 wherein the sensor is configured to detect a cerebral blood oxygen level.

70. The apparatus of claim 61 wherein the sensor is configured to detect a quantity that depends at least in part on a cerebral blood oxygen level.
